(19) United States
(12) Patent Application Publication
(10) Pub. No.: US 2020/0294669 A1
(43) Pub. Date: Sep. 17, 2020

(54) LEARNING METHOD, ESTIMATING METHOD, AND STORAGE MEDIUM

(71) Applicant: FUJITSU LIMITED, Kawasaki-shi (JP)

(72) Inventor: TAKESHI KONNO, Kawasaki (JP)

(73) Assignee: FUJITSU LIMITED, Kawasaki-shi (JP)

(21) Appl. No.: 16/812,433

(22) Filed: Mar. 9, 2020

(30) Foreign Application Priority Data
Mar. 11, 2019 (JP) 2019-043784
Sep. 5, 2019 (JP) 2019-161678

(51) Int. Cl. G16H 50/20; G06N 20/00; G06F 16/2458; G06N 5/04; G16H 50/30; G06K 9/62; G06K 9/00; A61B 5/00; A61B 5/11

(52) U.S. Cl. CPC G16H 50/20; G06N 20/00; G06F 16/2474; G06N 5/04; G16H 50/30; G06K 9/6256; A61B 5/1117; A61B 5/7264; A61B 5/1116; A61B 5/7275; A61B 5/112; A61B 5/4088; G06K 9/00348

(57) ABSTRACT

A learning method performed by a computer the learning method includes obtaining a posture of a target person and characteristics of external factors of the target person; generating a time series model related to the characteristics of the external factors based on the obtained characteristics of the external factors at different time points; and determining whether the target person has a disorder of an internal factor based on the obtained posture and the generated time series model.

### CROSS-REFERENCE TO RELATED APPLICATION

This application is based upon and claims the benefit of priority of the prior Japanese Patent Application Nos. 2019-161678, filed on Sep. 5, 2019, and 2019-43784, filed on Mar. 11, 2019, the entire contents of which are incorporated herein by reference.

### FIELD

The embodiments discussed herein are related to a learning method, an estimating method, and a storage medium.

### BACKGROUND

A risk of an elderly person is evaluated by performing determination of a locomotive syndrome indicating a state of a high risk of needing nursing care due to a disorder of a locomotive organ.

For the determination of this locomotive syndrome, the related art is known which calculates, for each given region of a subject, an acceleration of movement of each region, and determines a motor function using a neural network from a characteristic parameter obtained for each region from the calculated acceleration. Disclosed as the related art is, for example, Japanese Laid-open Patent Publication No. 2016-144598 or the like.

### SUMMARY

According to an aspect of the embodiments, a learning method includes obtaining a posture of a target person and characteristics of external factors of the target person; generating a time series model related to the characteristics of the external factors based on the obtained characteristics of the external factors at different time points; and determining whether the target person has a disorder of an internal factor based on the obtained posture and the generated time series model.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a block diagram illustrating an example of functional configuration of a learning device and an estimating device according to an embodiment;

FIG. 2 is an explanatory diagram illustrating user information;

FIG. 3 is an explanatory diagram of assistance in explaining generation of aging models;

FIG. 4 is an explanatory diagram of assistance in explaining posture determination;

FIG. 5 is an explanatory diagram of assistance in explaining modeling of aging models;

FIG. 6 is an explanatory diagram of assistance in explaining an example of regression analysis;

FIG. 7 is an explanatory diagram of assistance in explaining a dementia estimation;

FIG. 8 is an explanatory diagram of assistance in explaining a dementia estimation;

FIG. 9 is a flowchart illustrating an example of processing related to generation of an aging model;

FIG. 10 is a flowchart illustrating an example of processing related to a learning phase;

FIG. 11 is flowchart illustrating an example of processing related to an estimation phase;

FIG. 12 is an explanatory diagram illustrating an example of an output screen;

FIG. 13 is an explanatory diagram illustrating an example of comparison with a estimation result in an existing technique;

FIG. 14 is a flowchart illustrating an example of a modification of dementia determination;

FIG. 15 is an explanatory diagram of assistance in explaining an example of a dementia determination model;

FIG. 16 is an explanatory diagram of assistance in explaining determination example of a dementia determination model; and

FIG. 17 is a block diagram illustrating an example of a computer that executes a program.

### DESCRIPTION OF EMBODIMENTS

However, the above-described related art does not evaluate a potential risk of needing nursing care due to an internal factor such as dementia or the like, and may thus cause an erroneous determination. In view of the above, it is desirable to enhance the accuracy of estimation of a risk of needing nursing care.

A learning method, an estimating method, and a learning program according to embodiments will hereinafter be described with reference to the drawings. Configurations having the same functions in the embodiments are identified by the same reference numerals, and repeated description thereof will be omitted. It is to be noted that the learning method, the estimating method, and the learning program to be described in the following embodiments merely represent an example, and do not limit the embodiments. In addition, the following embodiments may be combined as appropriate within a scope where no inconsistency arises.

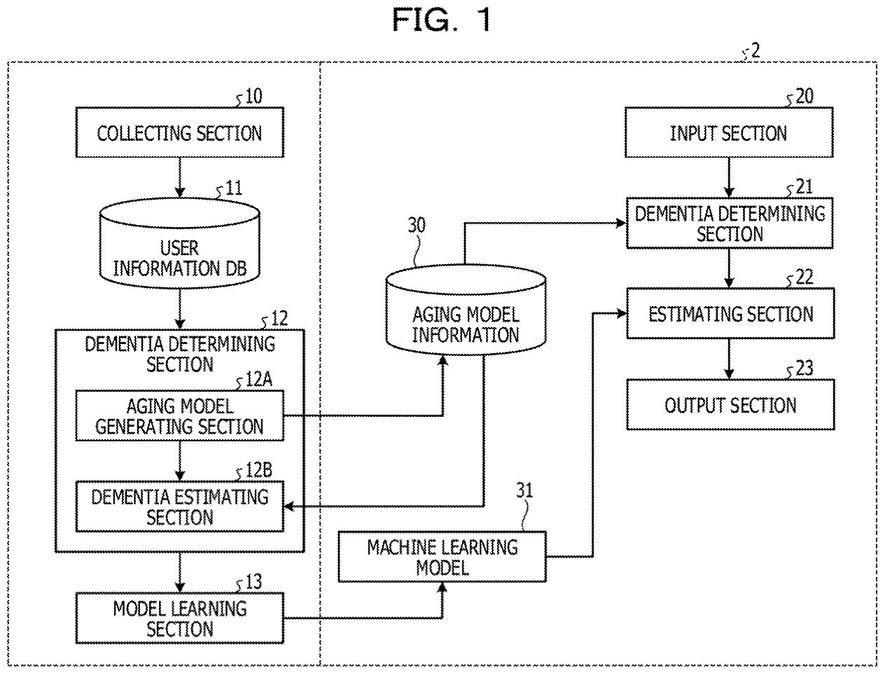

FIG. 1 is a block diagram illustrating an example of a functional configuration of a learning device and an estimating device according to an embodiment. As illustrated in FIG. 1, a learning device 1 is an information processing device that performs learning (supervised learning) of a machine learning model 31 for estimating a risk possessed by an estimation target person by using the characteristics of external factors of the estimation target person. For example, during learning (learning phase), the learning device 1 performs the learning of the machine learning model 31 by machine learning in which information including the characteristics of external factors of a learning target person as a teacher is set as an explanatory variable, and a risk as a correct answer in the learning target person is set as an objective variable.

In addition, an estimating device 2 is an information processing device that estimates a risk possessed by the estimation target person by using the machine learning model 31 learned by the learning device 1 and using the characteristics of external factors of the estimation target person. For example, the estimating device 2 estimates the risk possessed by the estimation target person by applying information including the characteristics of the external factors of the estimation target person to the learned machine learning model 31 during estimation (estimation phase).

In the present embodiment, description will be made by using as an example, a case where the characteristics of the external factors of the estimation target person are a mode (posture, a walking manner, or the like) during walking of the estimation target person, and a risk of needing nursing care due to falling or the like (falling risk) is estimated. Incidentally, the characteristics of the external factors are not limited to the mode during walking, but may be for example a mode during movement other than walking or the like. In addition, the risk to be estimated may be a risk (for example a risk of suffering from dementia) other than the falling risk.

The learning device 1 includes a collecting section 10, a user information database (DB) 11, a dementia determining section 12, and a model learning section 13.

The collecting section 10 is a processing section that collects information (age, the characteristics of external factors, a risk as a correct answer determined by an expert or the like, and the like) regarding the learning target person as a teacher of the machine learning model 31 through an operating input, a file input, or the like by a user. The collecting section 10 stores the collected user information of each learning target person in the user information DB 11. For example, the collecting section 10 stores, in the user formation DB 11, user information collected at each of a plurality of time points through periodic medical checkups or the like for each piece of identification information (user identification (ID) or the like) identifying a learning target person.

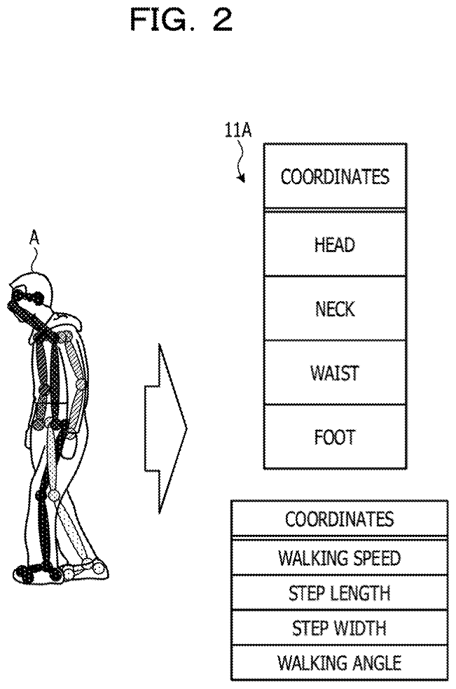

FIG. 2 is an explanatory diagram illustrating user information. As illustrated in FIG. 2, user information 11A is information indicating the characteristics of external factors of a target person A, and is information indicating a mode (posture, a walking manner, or the like) of the target person A at a time of walking in the present embodiment. For example, the user information 11A has coordinates of a “head,” a “neck,” a “waist,” and a “foot” indicating the posture of the target person A at a time of walking. In addition, the user information 11A has coordinates of a “walking speed,” a “step length,” a “step width,” and a “walking angle” indicating the walking manner of the target person A. In addition, the user information 11A is provided with the age of the target person A at a time of collection of the user information 11A, a correct answer label indicating a result of determination of a risk by an expert or the like (presence or absence of a falling risk or a risk degree), and the like in addition to the above-described information indicating the characteristics of the external factors of the target person A.

The dementia determining section 12 is a processing section that determines whether or not each learning target person has a disorder of an internal factor (dementia) based on the user information 11A of each learning target person, the user information 11A being stored in the user information DB 11. For example, the dementia determining section 12 includes an aging model generating section 12A and a dementia estimating section 12B.

The aging model generating section 12A is a processing section that generates time series aging models with regard to the characteristics of the external factors from the user information 11A collected at each of a plurality of time points by periodic medical checkups or the like for each learning target person. For example, the aging model generating section 12A generates an aging model indicating time series changes (changes accompanying aging) from data at the plurality of time points for each of the “walking speed,” the “step length,” the “step width,” and the “walking angle” indicating the walking manner in the user information 11A.

Incidentally, the characteristics of the external factors of the walking manner or the like differ greatly depending on the posture of the learning target person. Therefore, the aging model generating section 12A determines the posture of the learning target person based on the coordinates of the “head,” the “neck,” the “waist,” and the “foot” indicating the posture of the user information 11A.

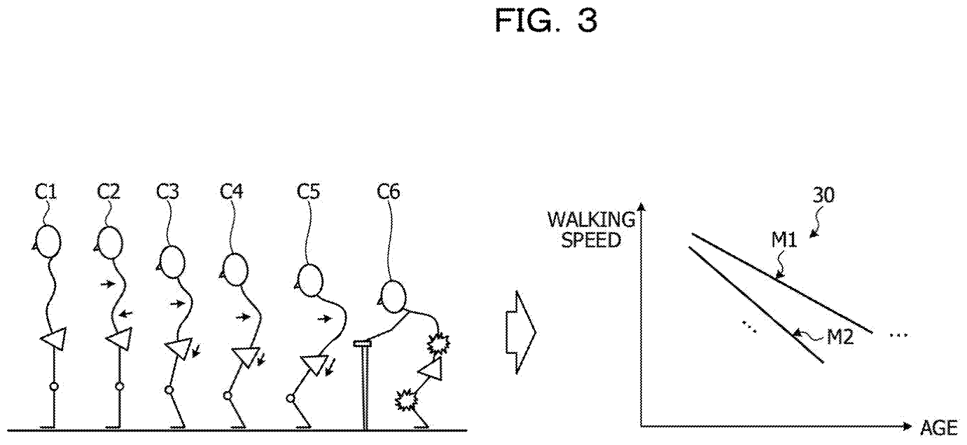

FIG. 3 is an explanatory diagram of assistance in explaining generation of aging models. As illustrated in FIG. 3, there are various postures such as postures C1 to C6 during walking of humans depending on, for example, physiological curves of the spine or the like. For example, the physiological curves of the spine include a deformation due to an osteoporotic vertebral body fracture, an intervertebral disk degeneration around a lumbar portion, or the like. In addition, there is a case where the posture is distorted in order to achieve a balance due to the pain of a knee.

Changes accompanying aging in the characteristics of the external factors (for example, the walking speed) differ for each of such postures C1 to C6. For example, the posture C1 changes as in an aging model M1, and the posture C2 changes as in an aging model M2.

Hence, the aging model generating section 12A determines which of the postures C1 to a the posture of the learning target person is based on the coordinates of the “head,” the “neck,” the “waist,” and the “foot,” and generates an aging model based on the determined posture.

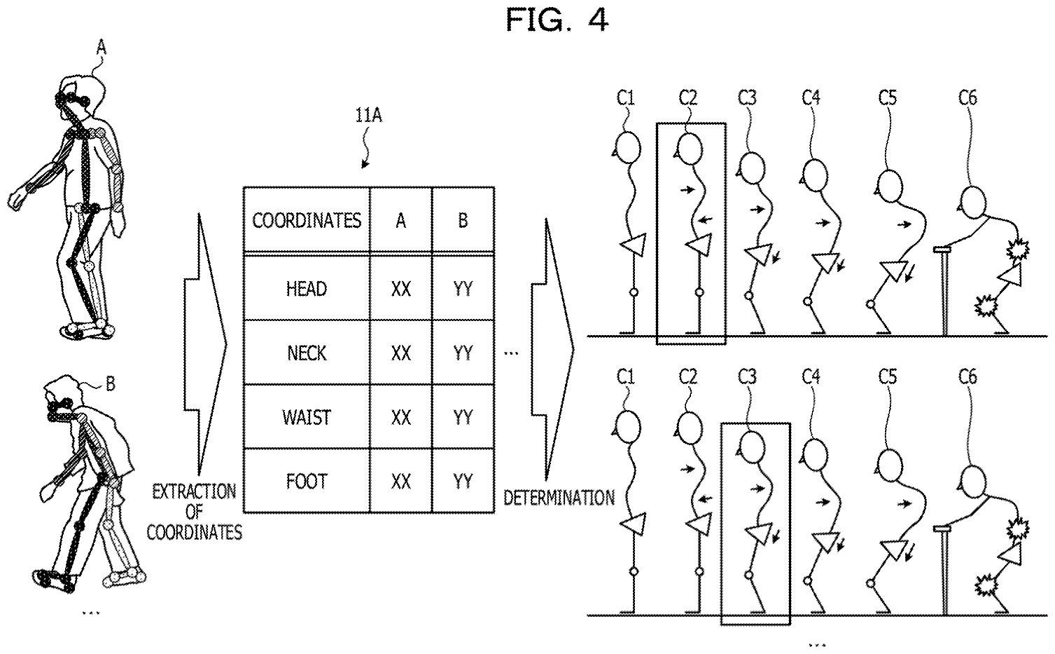

FIG. 4 is an explanatory diagram of assistance in explaining posture determination, As illustrated in FIG. 4, the aging model generating section 12A determines the postures of target persons A, B, . . . , based on which conditions of the postures C1 to C6 the coordinates of the “heads,” the “necks,” the “waists,” and the “feet” of the target persons A, B, . . . , in the user information 11A match. In the illustrated example, the target person A is determined to have the posture C2, and the target person B is determined to have the posture C3.

Next, for each learning target person, the aging model generating section 12A generates aging models in the determined posture by arranging and modeling the characteristics of external factors of the learning target person in time series order.

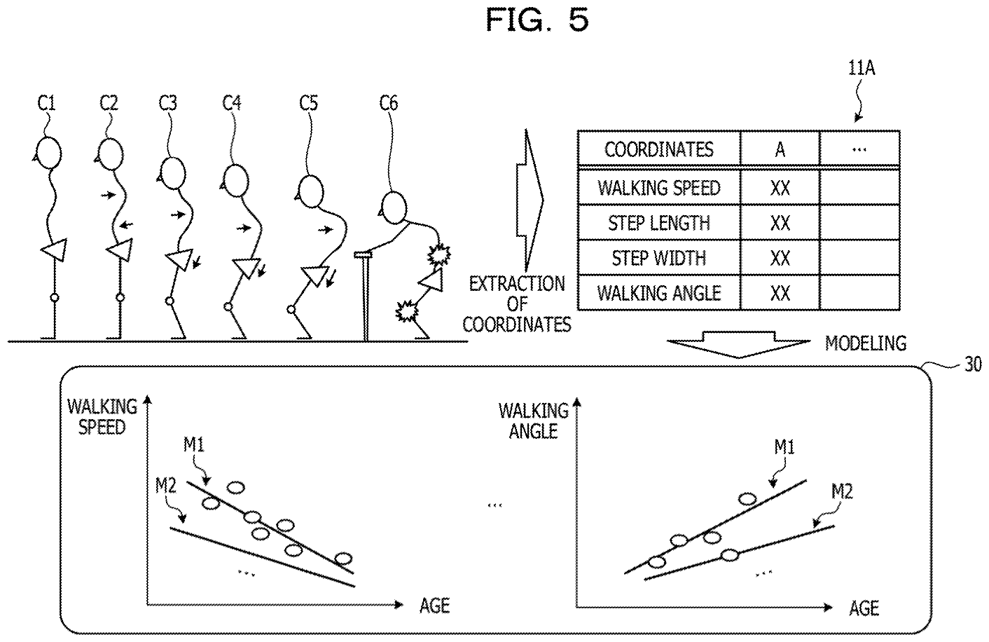

FIG. 5 is an explanatory diagram of assistance in explaining modeling of aging models. As illustrated in FIG. 5, the aging model generating section 12A generates aging models M1, M2, . . . , of each of the “walking speed,” the “step length,” the “step width,” and the “walking angle” indicating the walking manner in the user information 11A for the target persons A, B, . . . . For example, the aging model generating section 12A obtains the aging models M1, M2, . . . , by performing regression analysis of values obtained by plotting each of the “walking speed,” the “step length,” the “step width,” and the “walking angle” in time series order (age order).

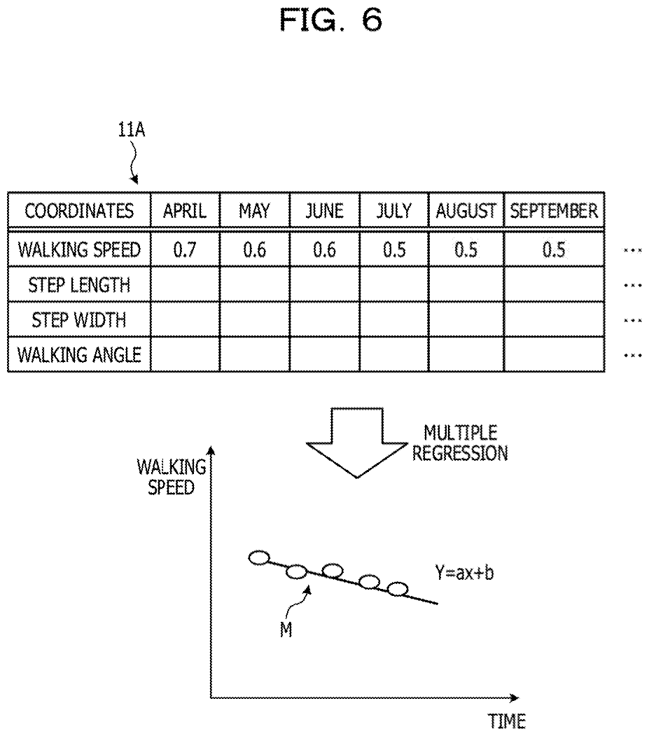

FIG. 6 is an explanatory diagram of assistance in explaining an example of regression analysis. As illustrated in FIG. 6, the aging model generating section 12A plots the walking speed obtained from April to September in time series order in the user information 11A. Next, the aging model generating section 12A performs multiple regression based on the plotted values, and thereby obtains an aging model M related to the walking speed in a form of Y=ax+b. Incidentally, in the following description, when aging models are not distinguished for each target person or are not distinguished for each of the “walking speed,” the “step length,” the “step width,” and the “walking angle,” the aging models will be referred to as aging models M.

Next, the aging model generating section 12A assigns identification information corresponding to the target persons to the obtained aging models 14, and then stores the aging models 14 as aging model information 30 in a memory or the like. Thus, in a learning phase and an estimation phase, the aging model M of each target person, may be obtained by referring to the aging model information 30 based on the identification information of the target person.

Returning to FIG. 1, the dementia estimating section 12B is a processing section that determines for each learning target person whether or not the target person has a disorder of an internal factor (dementia) based on the obtained posture and the generated aging model M.

The disorder of an internal factor (dementia in the present embodiment) of the target person appears as a significant change in time series in the characteristics of external factors by posture. For example, in a case where the target person has vascular dementia, the dementia progresses stepwise. A significant difference therefore occurs in the aging model M of the target person with respect to an aging model as a reference (hereinafter a reference model) according to aging for each posture. In addition, Lewy body dementia repeats a good condition and a bad condition, and therefore fluctuations with a given width in time series occur in the aging model M of the target person. The dementia estimating section 12B determines whether or not the target person has a disorder of an internal factor (dementia) based on such time series characteristics of the aging model M of the target person.

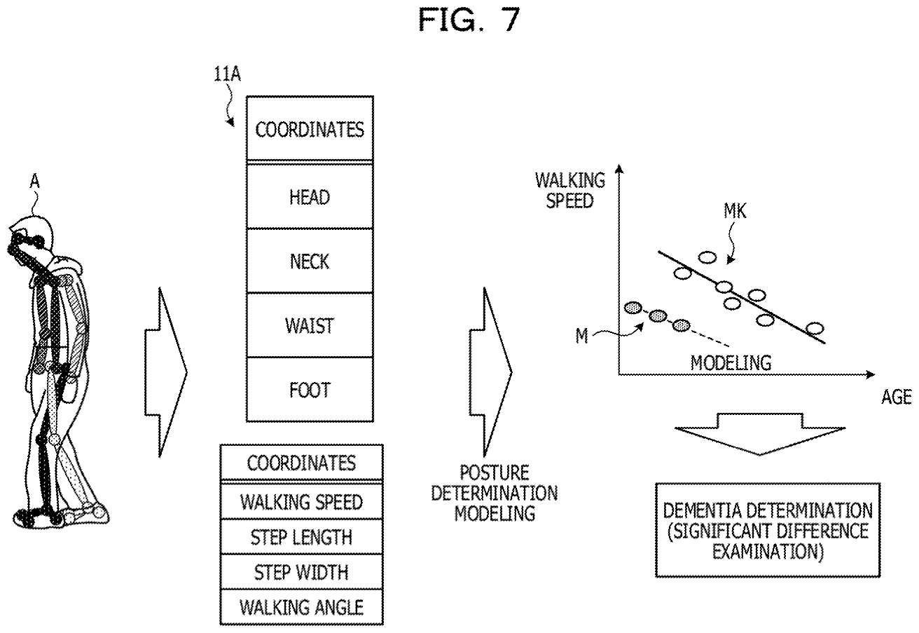

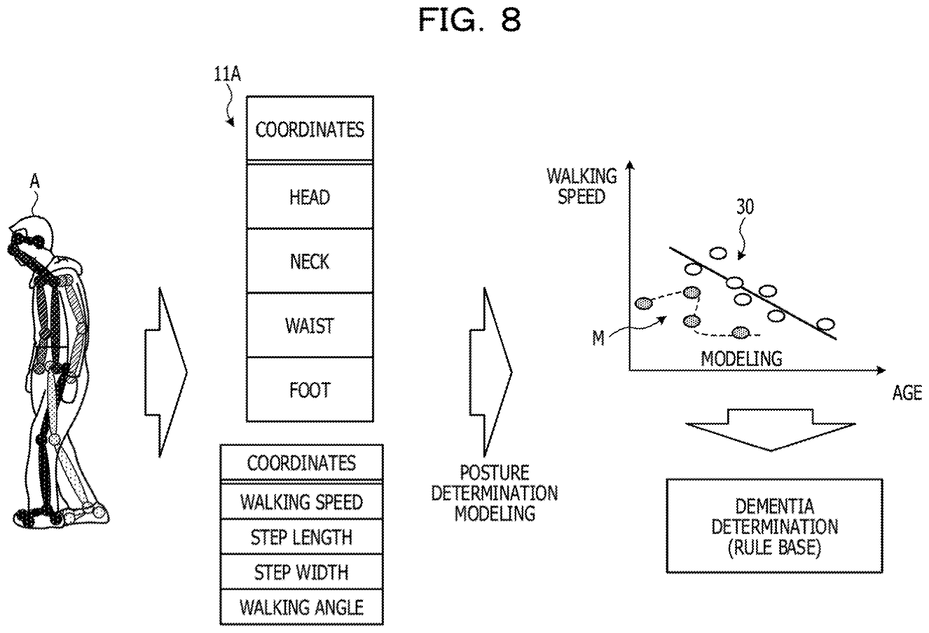

FIG. 7 and FIG. 8 are explanatory diagrams of assistance in explaining dementia estimation. For example, as illustrated in FIG. 7, the dementia estimating section 12B reads a reference model MK corresponding to the posture of the target person A from among reference models MK for respective postures, the reference models MK being preset in a memory or the like, based on the, posture determined from the user information 11A of the target person A. Incidentally, for the reference model MK, data set in the memory or the like in advance may be used, or an average of a large number of target persons, the average being stored in the aging model information 30, may be used.

Next, the dementia estimating section 12B performs statistical significant difference examination based on the reference model MK and the aging model M generated for the walking manner (the “walking speed,” the “step length,” the “step width,” and the “walking angle”) of the target person A. Next, the dementia estimating section 12B determines that the target person A has dementia when there is a significant difference between the reference model MK and the aging model M in the significant difference examination, and determines that the target person A has no dementia when there is no significant difference between the reference model MK and the aging model M in the significant difference examination.

In addition, as illustrated in FIG. 8, the dementia estimating section 12B determines, in a rule-based manner, presence or absence of fluctuation in a given range in time series for the aging model M generated for the walking manner (the “walking speed,” the “step length,” the “step width,” and the “walking angle”) of the target person A. Next, the dementia estimating section 12B determines that the target person A has dementia when there is fluctuation in the given range in time series, and determines that the target person A does not have dementia when there is no fluctuation in the given range in time series.

Returning to FIG. 1, the model learning section 13 is a processing section that performs learning of the machine learning model 31. For example, the model learning section 13 performs the learning of the machine learning model 31 by machine learning in which the presence or absence of a disorder of an internal factor (dementia), the presence or absence thereof being determined by the dementia determining section 12, and the user information 11A indicating the characteristics of external factors of the target person are set as an explanatory variable, and a risk given as a correct answer label is set as an objective variable.

Incidentally, applicable as the machine learning model 31 is, for example, a neural network in which units imitation the neurons of a brain are hierarchically coupled in a range from an input layer through a middle layer to an output layer.

The model learning section 13, for example, inputs the value of the objective variable to the input layer of the machine learning model 31, and makes an output value indicating an operation result output from the output layer. Then, the model learning section 13 learns a parameter at each node of the neural network in the machine learning model 31 based on comparison between the risk of the correct answer label and the output value. For example, the model learning section 13 learns the parameters of the neural network by an error back propagation (BP) method using a result of comparison between the output value and the correct answer label or the like.

The estimating device 2 includes an input section 20, a dementia determining section 21, an estimating section 22, and an output section 23.

The input section 20 is a processing section that receives input of information regarding an estimation target person (age, the characteristics of external factors, and the like) through an operating input, a file input, or the like by the user. The input section 20 outputs the received input information regarding the estimation target person to the dementia determining section 21.

The dementia determining section 21 is a processing section that determines whether or not the estimation target person has a disorder of an internal factor (dementia) based on the information regarding the estimation target person. Incidentally, the processing in the dementia determining section 21 is the same as that of the dementia determining section 12, though there is a difference between the learning target person and the estimation target person as a processing target. For example, the dementia determining section 21 has a configuration similar to that of the aging model generating section 12A and the dementia estimating section 12B. Hence, description of processing contents of the dementia determining section 21 will be omitted.

The estimating section 22 is a processing section that estimates a risk possessed by the estimation target person by using the learned machine learning model 31 in which the presence or absence of the disorder of the internal factor and the characteristics of external factors are set as an explanatory variable, and the risk is set as an objective variable. For example, the estimating section 22 estimates the risk of the estimation target person by applying the presence or absence of the disorder of the internal factor, the presence or absence thereof being determined by the estimating section 22, and the characteristics of the external factors of the estimation target person to the machine learning model 31. As an example, in a case where the machine learning model 31 is a neural network, the estimating section 22 inputs a value corresponding to the presence or absence of the disorder of the internal factor and the characteristics of the external factors to the input layer of the machine learning model 31, and obtains an output value (estimated risk) indicating an operation result from the output layer.

The output section 23 is a processing section that outputs an estimation result of the estimating section 22 by, for example, display output to a display, file output, or the like. The user may know the risk possessed by the estimation target person based on the output from the output section 21

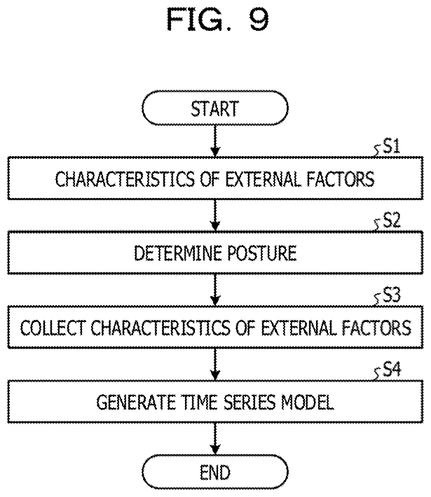

FIG. 9 is a flowchart illustrating an example of processing related to generation of an aging model. As illustrated in FIG. 9, when the processing is started, the aging model generating section 12A collects the characteristics of external factors in a learning target person (coordinates of the “head,” the “neck,” the “waist,” and the “foot”) from the user information DB 11 (S1).

Next, the aging model generating section 12A determines the posture of the learning target person based on the collected characteristics of the external factors (coordinates of the “head,” the “neck,” the “waist,” and the “foot”) (S2).

Next, the aging model generating section 12A collects data at a plurality of time points with regard to the characteristics of the external factors (the “walking speed,” the “step length,” the “step width,” and the “walking angle” indicating the walking manner) in the learning target person from the user information DB 11 (S3).

Next, the aging model generating section 12A generates a time series model (aging model M) from the data at the plurality of time points for each of the “walking speed,” the “step length,” the “step width,” and the “walking angle” indicating the walking manner (S4). Next, the aging model generating section 12A assigns identification information corresponding to the learning target person to the generated aging model M, and thereafter stores the aging model M in the aging model information 30. The aging model generating section 12A then ends the processing.

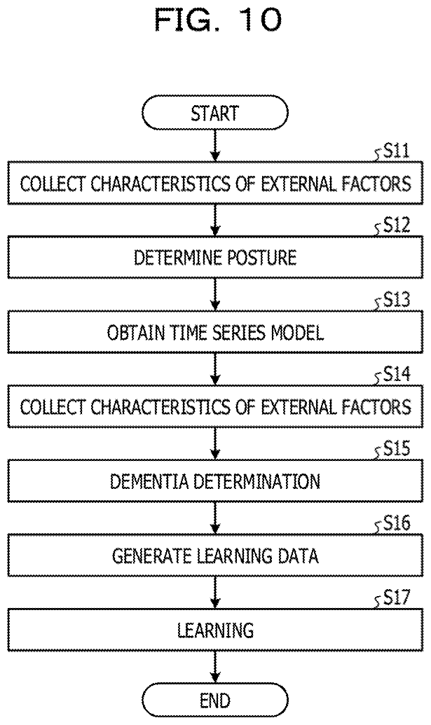

FIG. 10 is a flowchart illustrating an example of processing related to a learning phase. As illustrated in FIG. 10, when the processing related to the learning phase is started, the dementia estimating section 12B collects the characteristics of external factors (coordinates of the “head,” the “neck,” the “waist,” and the “foot”) in a learning target person from the user information DB 11 (S11).

Next, the dementia estimating section 12B determines the posture of the learning target person based on the collected characteristics of the external factors (coordinates of the “head,” the “neck,” the “waist,” and the “foot”) (S12). Next, the dementia estimating section 12B obtains a time series model (aging model M) to which identification information corresponding to the learning target person is assigned from the aging model information 30 (S13).

Next, the dementia estimating section 12B collects data at a plurality of time points with regard to the characteristics of external factors (the “walking speed,” the “step length,” the “step width,” and the “walking angle” indicating the walking manner) in the learning target person (S14).

Next, the dementia estimating section 12B determines whether or not the learning target person has a disorder of an internal, factor (dementia) based on the determined posture and the aging model M of the learning target person (S15).

Next, the model learning section 13 generates training data in which the presence or absence of the disorder of the internal factor (dementia), the presence or absence thereof being determined by the dementia determining section 12, and the characteristics of the external factors of the learning target person are set as an explanatory variable, and a risk given as a correct answer label is set as an objective variable (S16). Next, the model learning section 13 performs learning of the, machine learning model 31 by machine learning based on the generated training data (supervised learning) (S17).

The learning device 1 proceeds with the learning of the machine learning model 31 by performing the above-described processing of S11 to S17 on each learning target person in the learning phase.

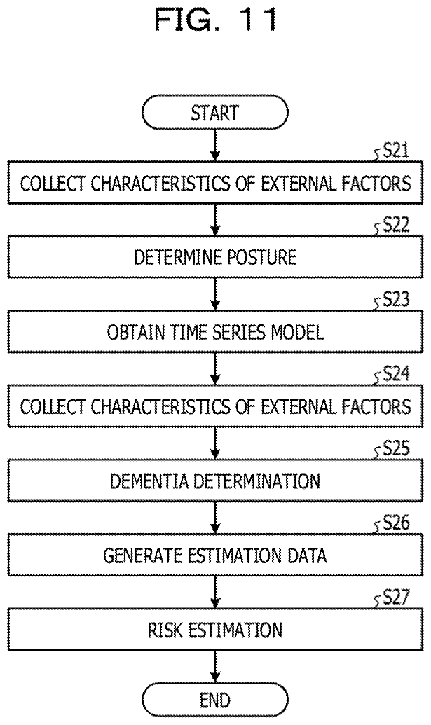

FIG. 11 is a flowchart illustrating an example of processing related to an estimation phase. As illustrated in FIG. 11, when the processing related to the estimation phase is started, the dementia determining section 21 collects the characteristics of external factors (coordinates of the “head,” the “neck,” the “waist,” and the “foot”) in an estimation target person (S21).

Next, the dementia determining section 21 determines the posture of the estimation target person based on the collected characteristics of the external factors (coordinates of the “head,” the “neck,” the “waist,” and the “foot”) (S22).

Next, tine dementia determining section 21 obtains a time series model (aging model M) to which identification information corresponding to the estimation target person is assigned, from the aging model information 30 (S23). Next, the dementia determining section 21 collects data at a plurality of time points with regard to the characteristics of the external factors (the “walking speed,” the “step length,” the “step width,” and the “walking angle” indicating the walking manner) in the estimation target person (S24).

Next, the dementia determining section 21 determines whether or not the estimation target person has a disorder of an internal factor (dementia) based on the determined posture and the aging model M of the estimation target person (S25).

Next, the estimating section 22 generates estimation data including the presence or absence of the disorder of the internal factor (dementia), the presence or absence thereof being determined by the dementia determining section 21, and the characteristics of the external factors of the estimation target person (S26), Next, the estimating section 22 estimates a risk of the estimation target person by applying the estimation data to the machine learning model 31 (S27). Next, the output section 23 outputs an estimation result of the estimating section 22 on a display screen or the like. The output section 23 then ends the processing.

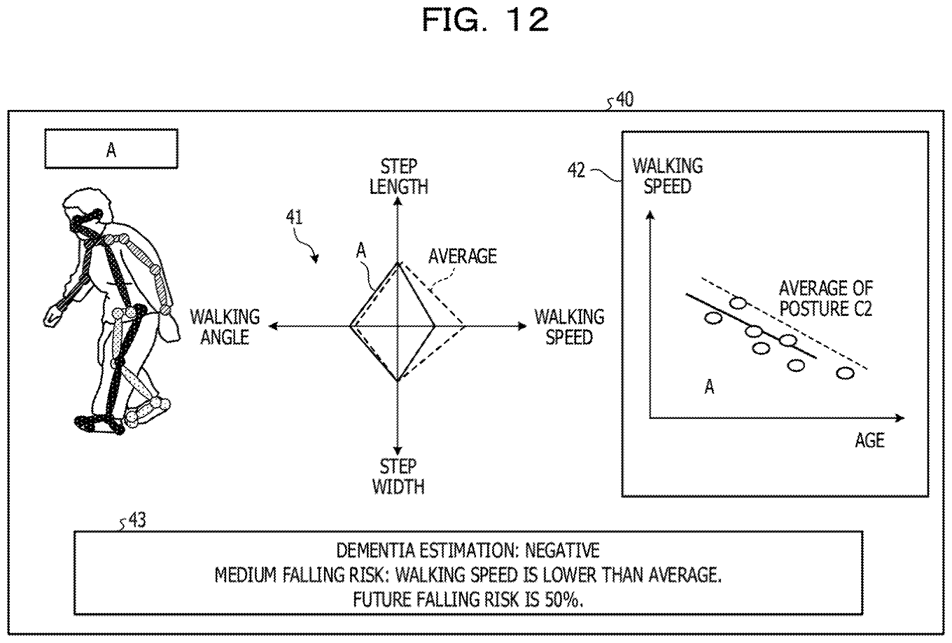

FIG. 12 is an explanatory diagram illustrating an example of an output screen. As illustrated in FIG. 12, an output screen 40 output by the output section 23 includes a walking manner display region 41 a model display region 42, and an estimation result display region 43.

The walking manner display region 41 is a display region indicating the “walking speed,” the “step length,” the “step width,” and the “walking angle” of the target person A and an overall average of each value. The user may readily know the walking manner (the “walking speed,” the “step length,” the “step width,” and the “walking angle” of the target person A) based on the contents displayed in the walking manner display region 41.

The model display region 42 is a region that displays an aging model M of the target person A and a reference model MK corresponding to the posture of the target person A (the posture C2 in the illustrated example). The user may readily compare the aging model M of the walking manner of the target person A with the reference model MK corresponding to the posture of the target person A based on the contents displayed in the model display region 42.

The estimation result display region 43 is a region that displays a result of determination of a disorder of an internal factor (dementia) by the dementia determining section 21 and an estimation result of the estimating section 22 for the target person A. The user may readily check whether or not the target person A has the disorder of the internal factor and the estimation result of a falling risk based on the contents displayed in the estimation result display region 43.

Description will next be made of a modification of dementia determination. In the above-described dementia determination, whether or not the target person has a disorder of an internal factor (dementia) is determined based on the walking manner (the “walking speed,” the “step length,” the “step width,” and the “walking angle”) as the characteristics of external factors in the target person.

However, there is a case where the walking speed is increased even when the target person has the disorder of the internal factor (dementia). In such a case, the target person may be erroneously determined not to have dementia, and thus accuracy of estimation of a potential risk of needing nursing care due to an internal factor such as dementia or the like is decreased.

In addition, the characteristics of external factors in the target person may appear in a manner of having meals or a conversation in addition to the walking manner. For example, when dementia develops, choking due to erroneous deglutition may occur as a result of putting food into the mouth indifferently without swallowing the food. It is difficult to estimate the potential risk of needing nursing care due to such erroneous deglutition from the characteristics of external factors of the walking manner or the like.

In a modification of the dementia determination, dementia determination is performed based on the characteristics of external factors other than the walking manner by using dementia logic different from the above-described dementia determination in the processing of S15 and S25 that perform the dementia determination. This may enhance accuracy of estimation of the potential risk of needing nursing care due to an internal factor such as dementia or the like.

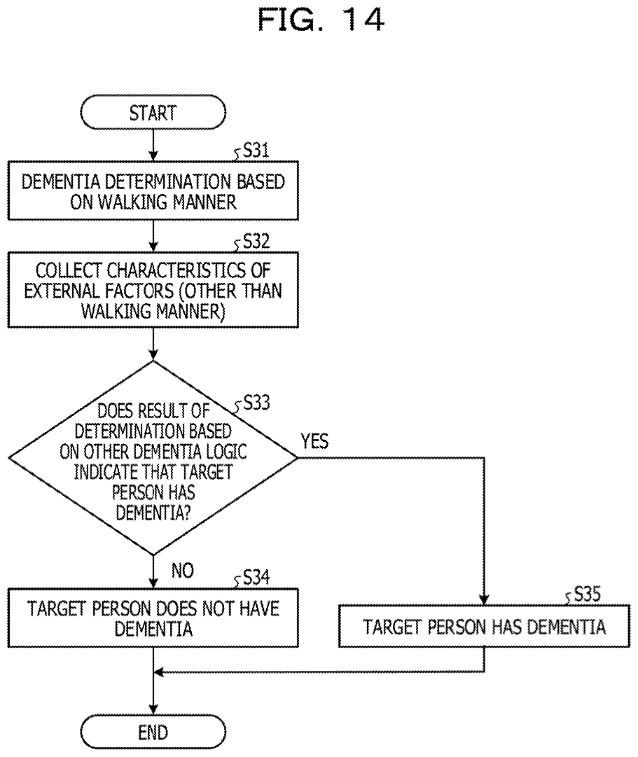

FIG. 14 is a flowchart illustrating an example of a modification of dementia determination. As illustrated in FIG. 14, when the processing of performing dementia determination is started, the dementia estimating section 12B first performs dementia determination based on the walking manner, the dementia determination being similar to S15 described above and the like (S31).

Next, the dementia estimating section 12B collects data at a plurality of time points with regard to the characteristics of external factors (other than the walking manner) in the target person from the user information DB 11 (S32). Incidentally, the characteristics of the external factors (other than the walking manner), for example, include items such as a “meal speed,” a “conversation speed,” “repeatedly saying the same thing,” “forgetting meals,” and the like. Suppose that as for these characteristics of the external factors (other than the walking manner), data collected from each user is registered in the user information DB 11 in advance.

Next, the dementia estimating section 12B performs dementia determination using a dementia determination model by dementia logic different from S15 and the like based on the characteristics of the external factors (other than the walking manner), the characteristics being collected for the target person (S33).

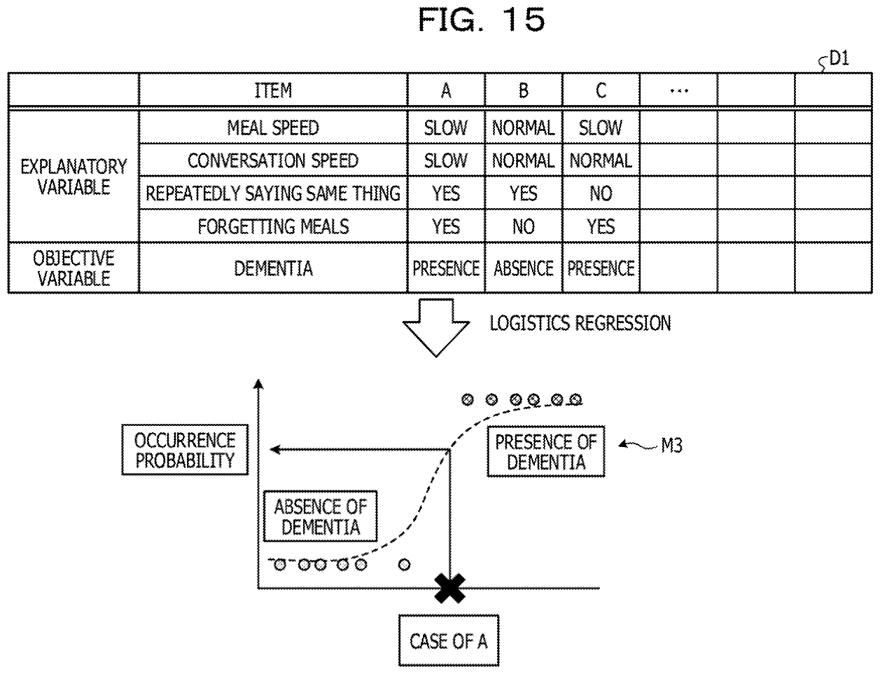

FIG. 15 is an explanatory diagram of assistance in explaining example of a dementia determination model. As illustrated in FIG. 15, a dementia determination model M3 that determines the presence or absence of dementia as a likelihood (occurrence probability) is obtained by logistics regression based on sample data D1 collected from each user.

For example, items such as the “meal speed,” the “conversation speed,” “repeatedly saying the same thing,” “forgetting meals,” and the like are set as an explanatory variable, the items being the characteristics of external factors (other than the walking manner), the characteristics being included in sample data D1 of each user. In addition, the presence or absence of dementia, the presence or absence thereof being included in the sample data D1 of each user, is set as an objective variable. Then, a regression coefficient, a constant term, and the like related to the dementia determination model M3 are determined by performing logistics regression analysis of the objective variable and the explanatory variable.

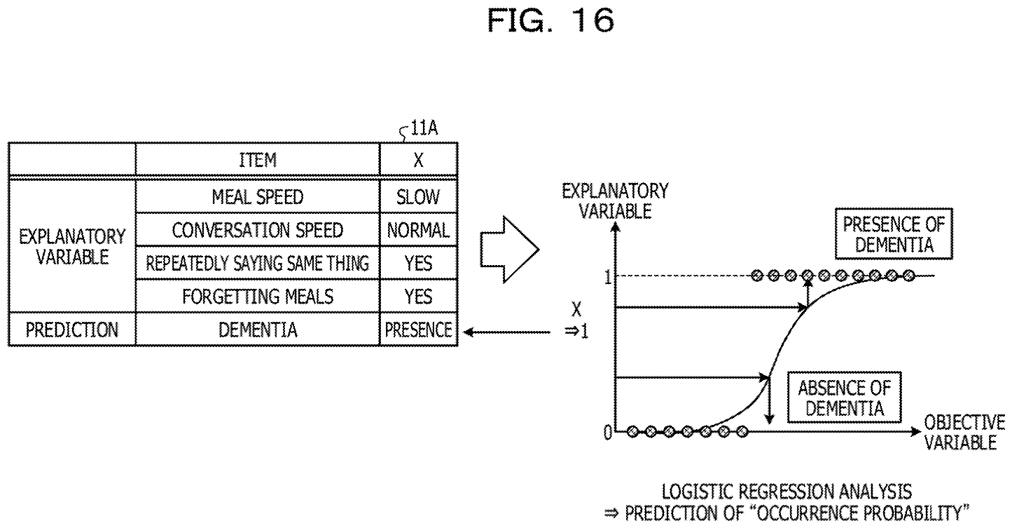

FIG. 16 is an explanatory diagram of assistance in explaining an example of determination of a dementia determination model. As illustrated in FIG. 16, the dementia estimating section 12B determines the presence or absence of dementia by applying the user information 11A of a target person to the dementia determination model M3. For example, the dementia estimating section 12B applies, to the dementia determination model M3, the items of the explanatory variable such as the “meal speed,” the “conversation speed,” “repeatedly saying the same thing,” “forgetting meals,” and the like in the user information 11A of the target person. The dementia estimating section 12B may thereby obtain the presence or absence of dementia as a likelihood (occurrence probability, for example). The dementia estimating section 12B determines the presence or absence of dementia based on the occurrence probability obtained for dementia (by comparing the occurrence probability with a given threshold value, for example).

Next, when the occurrence probability is lower than the threshold value in the dementia determination, for example, when there is no dementia (S33 absence), the dementia estimating section 12B determines that there is no dementia with regard to the characteristics of the external factors (S34). In addition, when the occurrence probability is higher than the threshold value in the dementia determination, for example, when there is dementia (S33: presence), the dementia estimating section 126 determines that there is dementia with regard to the characteristics of the external factors (S35).

Incidentally, the dementia estimating section 126 may treat a result of the dementia determination in S31 and a result of the dementia determination in S34 or S35 as respective different determination results, or may combine the results with each other.

For example, the dementia estimating section 12B may set the presence of dementia/absence of dementia in S31 as a first dementia determination result, and set the presence of dementia/absence of dementia in S34 or S35 as a second dementia determination result. In addition, the dementia estimating section 126 may determine that there is dementia when one of the first dementia determination result and the second dementia determination result indicates the presence of dementia, and may determine that there is no dementia when both of the first and second dementia determination results indicate the absence of dementia.

As described above, the aging model generating section 12A of the learning device 1 obtains a posture of a target person and characteristics of external factors of the target person, and generates an aging model M related to the characteristics of the external factors based on a plurality of the obtained characteristics of the external factors at different time points. The dementia estimating section 12B of the learning device 1 determines whether or not the target person has a disorder of an internal factor (dementia) based on the obtained posture and the generated aging model M. Thus, the learning device 1 may evaluate a potential risk of needing nursing care due to an internal factor such as dementia or the like.

In addition, the model learning section 13 of the learning device 1 performs learning, of the machine learning model 31 using determined presence or absence of the disorder of the internal factor and the obtained characteristics of the external factors as an explanatory variable, and using a risk possessed by the target person as an objective variable.

Thus, the learning device 1 adds the presence or absence of the disorder of the internal factor, the presence or absence thereof being determined for the target person, as an explanatory variable. The learning device 1 may therefore learn the machine learning model 31 so as to estimate the risk after taking into consideration a potential factor due to an internal factor such as dementia or the like. The accuracy of estimation of the risk may be consequently enhanced.

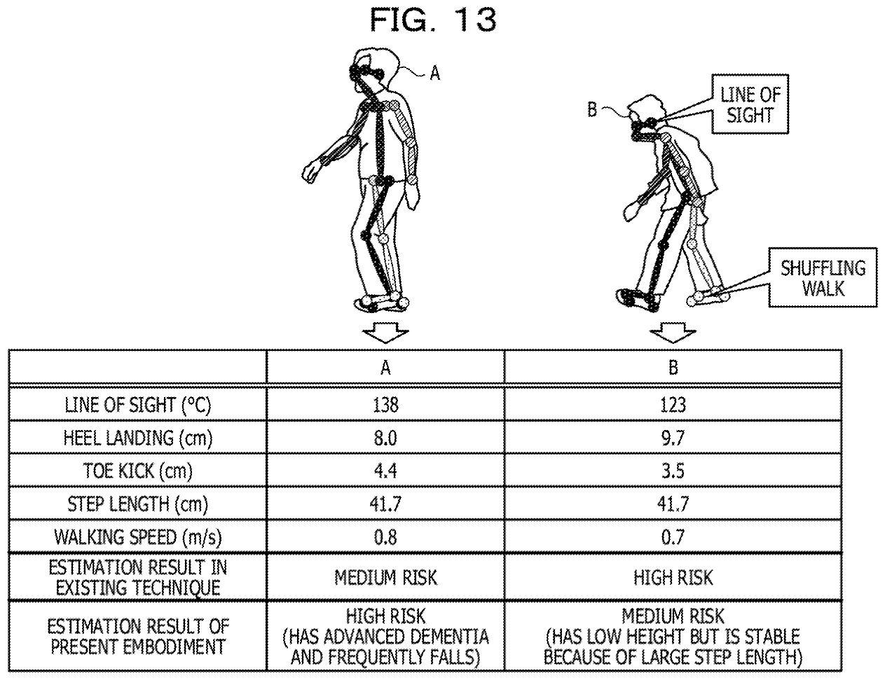

FIG. 13 is an explanatory diagram illustrating an example of comparison with a estimation result in an existing technique. As illustrated in FIG. 13, when the target persons A and B are compared with each other, the target person B who has a downward line of sight and shuffles appears to have a deformed posture, and therefore appears to have a higher risk. Therefore, because a potential falling risk due to an internal factor is not evaluated in the estimation result in an existing technique, the target person B is evaluated as having a higher risk than the target person A.

In actuality, though the target person A has an upright posture closer to a normal posture than the target person B, there is a significant difference between the aging model M of the target person. A and the reference model MK, and the target person A has dementia. In the present embodiment, a potential factor due to an internal factor such as dementia or the like is added to the explanatory variable, and the learning of the machine learning model 31 is performed. Therefore, an estimation result of the present embodiment indicates that the target person A having dementia has a higher risk than the target person B. The estimation result of the present embodiment is thus a more accurate estimation result.

In addition, the dementia estimating section 12B determines presence or absence of the disorder of the internal factor based on a comparison between a reference model corresponding to the obtained posture among reference models (M1, M2, . . . ) according to aging for respective postures and the generated aging model M. Thus, the learning device 1 may determine the presence or absence of the disorder of the internal factor by the comparison between the reference model according to aging, the reference model corresponding to the posture of the target person, and the aging model M of the target person. For example, when there is a significant difference between the reference model and the aging model M, it may be determined that the target person has the disorder of the internal factor.

In addition, the dementia estimating section 12B determines that the target person has the disorder of the internal factor when the characteristics of the external factors in the aging model M have fluctuation in time series. For example, the dementia estimating section 12B may determine that the target person has the disorder of the internal factor (dementia) when the walking speed has fluctuation having a given range such that a good condition and a bad condition are repeated.

In addition, the posture of the target person may be a posture during walking of the target person, and the characteristics of the external factors may be a walking manner of the target person. Thus, the learning device 1 may perform learning of the machine learning model 31 for estimating the risk possessed by the target person based on the posture during walking of the target person and the walking manner of the target person.

In addition, the disorder of the internal factor may be dementia. Thus, the learning device 1 may add the presence or absence of dementia as an explanatory variable in the learning of the, machine learning model 31 for estimating the risk possessed by the target person.

In addition, the dementia determining section 21 of the estimating device 2 obtains a posture of a target person and characteristics of external factors of the target person, and generates an aging model M related to the characteristics of the external factors based on a plurality of the obtained characteristics of the external factors at different time points. The dementia determining section 21 of the estimating device 2 determines whether or not the target person has a disorder of an internal factor (dementia) based on the obtained posture and the generated aging model M. The estimating section 22 of the estimating device 2 estimates a risk of the target person by applying determined presence or absence of the disorder of the internal factor and the obtained characteristics of the external factors to the machine learning model 31 learned by using the presence or absence of the disorder of the internal factor and the characteristics of the external factors as an explanatory variable and using the risk as an objective variable.

Thus, the estimating device 2 estimates the risk after taking into consideration a potential factor due to an internal factor such as dementia or the like by using the machine learning model 31. The accuracy of estimation of the risk may therefore be enhanced.

In addition, the dementia determining section 21 determines the presence or absence of the disorder of the internal factor based on a comparison between a reference model corresponding to the obtained posture among reference models (M1, M2, . . . ) according to aging for respective postures and the generated aging model M. Thus, the estimating device 2 may determine the presence or absence of the disorder of the internal factor by the comparison between the reference model according to aging, the reference model corresponding to the posture of the target person, and the aging model M of the target person. For example, when there is a significant difference between the reference model and the aging model M, it may be determined that the target person has the disorder of the internal factor.

In addition, the dementia determining section 21 determines that the target person has the disorder of the internal factor when the characteristics of the external factors in the aging model M have fluctuation in time series. For example, the dementia determining section 21 may determine that the target person has the disorder of the internal factor (dementia) when the walking speed has fluctuation having a given range such that a good condition and a bad condition are repeated.

In addition, the posture of the target person may be a posture during walking of the target person, and the characteristics of the external factors may be a walking manner of the target person. Thus, the estimating device 2 may estimate the risk possessed by the target person based on the posture during walking of the target person and the walking manner of the target person.

In addition, the disorder of the internal factor may be dementia. Thus, the estimating device 2 may estimate the risk possessed by the target person by applying the presence or absence of dementia to the machine learning model 31.

Incidentally, the constituent elements of the respective devices illustrated in the figures may not necessarily required to be physically configured as illustrated in the figures. For example, specific forms of distribution and integration of the devices are not limited to those illustrated in the figures, but the whole or a part of the devices may be configured so as to be distributed and integrated functionally or physically in arbitrary units according to various kinds of loads, usage conditions, and the like.

The whole or an arbitrary part of various kinds of processing functions performed in the learning device 1 and the estimating device 2 may be performed on a central processing unit (CPU) (or a microcomputer such as a micro processing unit (MPU), a micro controller unit (MCU), or the like). In addition, it is needless to say that the whole or an arbitrary part of the various kinds of processing functions may be performed on a program analyzed and executed by the CPU (or the microcomputer such as the MPU, the MCU, or the like) or on hardware based on wired logic. In addition, the various kinds of processing functions performed in the learning device 1 may be performed with a plurality of computers cooperating with each other by cloud computing.

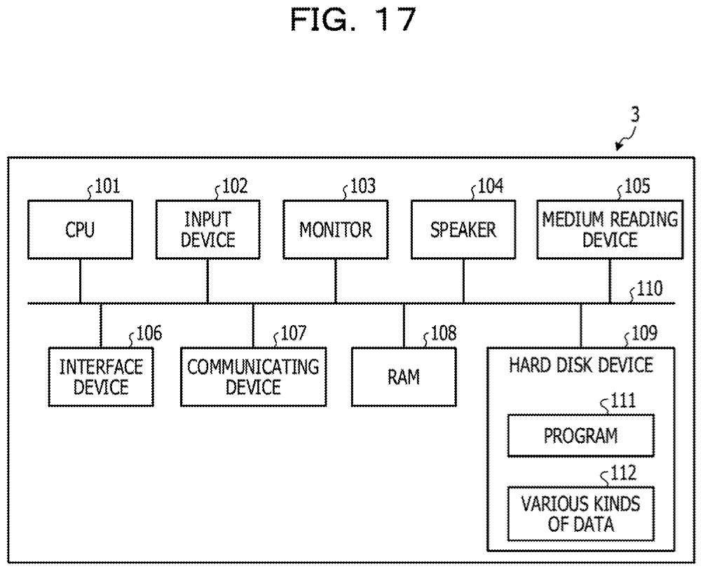

Various kinds of processing described in the foregoing embodiment may be implemented by executing a program prepared in advance on a computer. Accordingly, the following description will be made of an example of a computer (hardware) that executes a program having functions similar to those of the foregoing embodiment. FIG. 17 is a block diagram illustrating an example of a computer that executes a program.

As illustrated in FIG. 17, a computer 3 includes a CPU 101 that executes various kinds of arithmetic processing, an input device 102 that receives data input, a monitor 103, and a speaker 104. The computer 3 also includes a medium reading device 105 that reads a program or the like from a storage medium, an interface device 106 for coupling with various kinds of devices, and a communicating device 107 for communication coupling with an external apparatus by wire or radio. The computer 3 also includes a random access memory (LAN) 108 that temporarily stores various kinds of information and a hard disk device 109. In addition, the units (101 to 109) within the computer 3 are coupled to a bus 110.

The hard disk device 109 stores a program 111 for performing various kinds of processing described in the foregoing embodiment. In addition, the hard disk device 109 stores various kinds of data 112 that the program 111 refers to. The input device 102, for example, receives an input of operation information from an operator of the computer 3. The monitor 103, for example, displays various kinds of screens operated by the operator. The interface device 106 is, for example, coupled with a printing device or the like. The communicating device 187 is coupled to a communication network such as a local area network (LAN) or the like, and, exchanges various kinds of information with an external apparatus via the communication network.

The CPU 101 performs various kinds of processing by reading the program 111 stored in the hard disk device 109, expanding the program 111 in the RAM 108, and executing the program 111. For example, in the case of the learning device 1 the CPU 101 performs processing related to the collecting section 10, the dementia determining section 12, and the model learning section 13 by executing the program 111. In addition, in the case of the estimating device 2, the CPU 101 performs processing related to the input section 20, the dementia determining section 21, the estimating section 22, and the output section 23 by executing the program 111.

Incidentally, the program 111 may not be stored in the hard disk device 109. For example, the program 111 stored on a storage medium readable by the computer 3 may be read and executed by the computer 3. The following, for example, correspond to the storage medium readable by the computer 3: a portable recording medium such as a compact disc-read only memory (CD-ROM), a digital versatile disk (DVD), a universal serial bus (USB) memory, or the like, a semiconductor memory such as a flash memory or the like, a hard disk drive, and the like. In addition, the program 111 may be stored in devices coupled to a public circuit, the Internet, a LAN, or the like, and the computer 3 may read the program 111 from these devices and execute the program 111.

All examples and conditional language provided herein are intended for the pedagogical purposes of aiding the reader in understanding the invention and the concepts contributed by the, inventor to further the art, and are not to be construed as limitations to such specifically recited examples and conditions, nor does the organization of such examples in the specification relate, to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A learning method performed by a computer the learning method comprising:
obtaining a posture of a target person and characteristic external factors of the target person;
generating a time series model related to the characteristics of the external factors based on the obtained characteristics of the external factors at different time points; and
determining whether the target person has a disorder of an internal factor based on the obtained posture and the generated time series model.

2. The learning method according to claim 1, further comprising
perform machine learning using determined presence or absence of the disorder of the internal factor and the obtained characteristics of the external factors as an explanatory variable, and using a risk possessed by the target person as an objective variable.

3. The learning method according to claim 1, wherein
the determining includes determining presence or absence of the disorder of the internal factor based on a comparison between a reference model corresponding to the obtained posture among reference models according to aging for respective postures and the generated model.

4. The learning method according to claim 3, wherein
the determining includes determining that the target person has the disorder of the internal factor when there is a significant difference between the reference model and the generated model.

5. The learning method according to claim wherein
the determining includes determining that the target person has the disorder of the internal factor when the characteristics of the external factors in the generated model have fluctuation in time series.

6. The learning method according to claim 1, wherein
the posture is a posture during walking of the target person, and
the characteristics of the external factors are a walking manner of the target person.

7. The learning method according to claim 1, wherein
the disorder of the internal factor is dementia.

8. An estimating method performed by a computer, the estimating method comprising:
obtaining a posture of a target person and characteristics of external factors of the target person;
generating a time series model related to the characteristics of the eternal factors based on a plurality of the obtained characteristics of the external factors at different time points;
determining whether the target person has a disorder of an internal actor based on the obtained posture and the generated model; and
estimating a risk of the target person by applying determined presence or absence of the disorder of the internal factor and the obtained characteristics of the external factors to a machine learning model learned by using the presence or absence of the disorder of the internal factor and the characteristics of the external factors as an explanatory variable, and using the risk as an objective variable.

9. A non-transitory computer-readable storage medium storing a program that causes a computer to execute a process, the process comprising:
obtaining a posture of a target person and characteristics of external factors of the target person;
generating a time series model related to the characteristics of the external factors based on the obtained characteristics of the external factors at different time points; and
determining whether the target person has a disorder of an internal actor based on the obtained posture and the generated time series model.

10. The storage medium according to claim 9, further comprising
Perform machine learning using determined presence or absence of the disorder of the internal factor and the obtained characteristics of the external factors as an explanatory variable, and using a risk possessed by the target person as an objective variable.

11. The storage medium according to claim 9, wherein
the determining includes determining presence or absence of the disorder of the internal factor based on a comparison between a reference model corresponding to the obtained posture among reference models according to aging for respective postures and the generated model.

12. The storage medium according to claim 11, wherein
the determining includes determining that the target person has the disorder of the internal factor when there is a significant difference between the reference model and the generated model.

13. The storage medium according to claim 11, wherein
the determining includes determining that the target person has the disorder of the internal factor when the characteristics of the external factors in the generated model have fluctuation in time series.

14. The storage medium according to claim 9 wherein
the posture is a posture during walking of the target person, and
the characteristics of the external factors are a walking manner of the target person.

15. The storage medium according to claim 1, wherein
the disorder of the internal factor is dementia.
